(19) United States
(12) Patent Application Publication
(10) Pub. No.: US 2009/0191249 A1
(43) Pub. Date: Jul. 30, 2009

(54) Sheet substrates impregnated with aromatic releasing compositions and a method of delivery of aromatic releasing compositions

(76) Inventor: Olufemi Adelakun, Hialeah, FL (US)

Correspondence Address: ROBERT M. DOWNEY, P.A., 6751 N. FEDERAL HWY., SUITE 300, BOCA RATON, FL, 33487, US

(21) Appl. No.: 12/012,047

(22) Filed: Jan. 30, 2008

(51) Int. Cl. A01N 25/34; A61K 9/70; A01P 15/00; A61P 29/00; A61P 31/00

(52) U.S. Cl. 424/405; 424/443

(57) ABSTRACT

An article of manufacture and a method directed to application of aromatic releasing compositions impregnated within substrates such as non-woven paper materials (e.g., wipes, paper towels) and dispensable cloth materials (e.g., gauze or a thin fabric of silk, linen, or cotton materials) for providing relief from cold, allergies, sinus and symptoms associated with respiratory disorders, the aromatic releasing compositions including the following: Menthol; Camphor; Eucalyptus oil; Cedarleaf Oil; Myristica Oil; Peppermint Oil; Lavender oil; Methyl Salicylate; Naproxen; Nutmeg Oil and Thymol; Beclometasone dipropionate; Benzethonium chloride with base solution consisting of Emollients, Emulsifiers and Moisturizer; Deionized Water; Vegetable Oil; Dicaprylyl Carbonate; Glyceryl Oleate; Polyglyceryl-2 Dipolyhydroxystearate; Cetearyl Isononanoate; Ceteareth-20; Cetearyl Alcohol; Glyceryl Stearate; Glycerin; Cetyl Palmitate; Ceteareth-12, Lauryl Glucose Carboxylate; Lauryl Glucoside; Sodium Citrate; Citric Acid; Benzethonium Chloride 0.05%; Ethylene diamine tetra acetic acid; Phenoxyethanol; Methylparaben; Propylparaben; 2-bromo-2-nitropropane-1,3-diol; and subcombinations thereof. In further embodiments, the compositions impregnated within substrate further include one or more topical actives, and are useful for providing relief from cold, allergies, sinus and symptoms associated with respiratory disorders, as well as repelling common virus and bacteria.

### BACKGROUND OF THE INVENTION

1. Field of the Invention

The present invention relates to paper wipe materials (i.e, non-woven) and dispensable cloth materials (e.g., gauze or a thin fabric of silk, linen, or cotton materials) impregnated with aromatic compounds, alone or in combination with oil-in-water emulsion pharmaceutical compositions for inhaled delivery to the user. In particular, the present invention relates to aromatic releasing compositions substantially impregnated in paper wipes or non-woven paper that are free from petrolatum, the aromatic releasing compositions containing one or more volatile aromatic compounds selected from the group consisting of: Menthol; Camphor; Eucalyptus oil; Cedarleaf Oil; Myristica Oil; Peppermint Oil; and mixtures thereof.

2. Discussion of the Related Art

It is well known that there is no cure for the common cold. At best, all that can be done is to reduce the period of infliction and alleviate the symptoms. Early symptoms of the common cold are usually minimal with only mild malaise, sore throat and nasal congestion. When the cold is a result of rhinovirus infection, symptoms of nasal discharge, nasal congestion, and sneezing usually begin on the first day of illness and progress to maximum severity by the second or third day. Other symptoms include sore throat and hoarseness and cough, as well as mild burning of the eyes, loss of smell and taste, a feeling of pressure or fullness in the sinuses or ears, headache, and vocal impairment. Influenza infection generally includes fever, often of sudden onset and persisting for several days, and with great severity; generalized aches and pains; fatigue and weakness; and chest discomfort.

As noted above, only symptomatic treatment is available for the common cold. Exemplary prior art oral compositions for treatment of cough, cold, cold-like and/or flu symptoms and the discomfort, pain, fever and general malaise associated therewith generally contain an analgesic (aspirin or acetaminophen) and one or more antihistamines, decongestants, cough suppressants, antitussives and expectorants. For individuals with certain medical conditions such as heart disease, hypertension, diabetes or thyroid disorders, oral drugs such decongestants could pose a risk of unfavorable drug interactions and may cause an adverse reaction. It has been previously suggested that it is highly desirable to deliver relief from these symptoms via topical compositions, without the need to orally ingest drugs. Such topical cold medications will not cause drowsiness or other side effects attendant with oral decongestants. Prior art topical compositions containing aromatic actives are effective at treating many of these symptoms, such as nasal congestion and cough.

An example of topical aromatic releasing compositions can be found in U.S. Pat. No. 5,322,689 to Hughes et al. The topical aromatic releasing compositions disclosed in this patent are provided in a gel form and are applied topically to the skin, preferably below the nose and on the chest. It has been found that, in some instances, topical application can be irritating to the skin. Moreover, applying a gel composition to the face is cosmetically unappealing. Further, the aroma of the topical aromatic releasing composition may be offensive to others in close proximity to a person wearing the composition on their face, chest or other areas on the body. Accordingly, there is an urgent and definite need for an improved product and method for inhaled delivery of aromatic releasing compositions without irritating the skin or annoying others. There is a further need for an improved product and method for inhaled delivery of aromatic releasing compositions that does not impair the user's cosmetic appearance.

It is, therefore, an object of the present invention to provide a means for convenient and effective inhaled delivery of aromatic releasing compositions to provide treatment for cough, cold, cold-like and/or flu symptoms. It is a further object of the present invention to provide non-woven paper wipes impregnated with aromatic releasing compositions as a vehicle of release and inhaled delivery of aromatic vapors. It is still a further object of the present invention to provide aromatic compositions for inhaled delivery from a non-woven paper wipe or dispensable cloth material which minimize the likelihood of adverse drug interactions and further which provide for proper medication management.

### SUMMARY OF THE INVENTION

The present invention relates to a wipe pregnanted with an aromatic decongestant composition that is substantially free from petrolatum. The wipe is preferably a non-woven paper material or a dispensable cloth material.

Non-woven materials are typically produced from man-made fibers. Two synthetic polymers dominate the market; polypropylene (PP) and polyesters (PET). Nonwovens are often application-designated as either durable or disposable. For example, nonwovens used as housewipes to prevent water infiltration are durable nonwovens. Nonwovens used as facings on baby diapers are disposable or single-use nonwovens.

The aromatic decongestant composition contains from about 0.05% to about 30% of one or more volatile aromatic compounds selected from the group consisting of: Menthol; Camphor and Eucalyptus oil; Cedarleaf Oil; Myristica Oil; Lavender oil; Thymol; Sodium Hydroxide; Disodium EDTA; Methylparaben; Isostearyl Benzoate; Propylparaben and mixtures thereof.

The present invention is also directed to a method for treatment of cough, cold, cold-like and/or flu symptoms comprising administering a safe and effective amount of these aromatic releasing decongestant compositions by placing the impregnated wipe on or in close proximity to the nose and face and breathing, to thereby inhale the composition.

All levels and ratios are by weight of the total composition, unless otherwise indicated.

### BRIEF DESCRIPTION OF THE DRAWINGS

For a fuller understanding of the nature of the present invention, reference should be made to the following detailed description taken in conjunction with the accompanying drawings in which:

FIG. 1 is a perspective view showing a plurality of non-woven paper substrate sheets, preferably wipes, folded and arranged in a stack for packaging and dispensing;

FIG. 2 is perspective view showing the stacked arrangement of non-woven paper wipes within a rigid tub container including a base and a removable lid;

FIG. 3 is a top perspective view showing another embodiment of packaging for the stacked arrangement of paper wipes of FIG. 1, wherein the package includes a liquid impervious flexible envelope including a peel-away re-sealable cover for opening the package to remove individual wipes and resealing the package closed after removal of one or more wipes;

FIG. 4 is a top perspective view of the package of FIG. 3 showing the peel-away cover pulled open to permit individual dispensing of the wipes contained therein;

FIG. 5 is top perspective view illustrating a further embodiment of a package for containing the stacked arrangement of wipes of FIG. 1 including a liquid impervious envelope with a hinged rigid cover operable between a closed, sealed position and an open position to permit individual removal of one or more wipes contained within the envelope package;

FIG. 6 is a top perspective view of the package of FIG. 5 shown with the rigid cover partially open to permit removal of one or more wipes contained therein;

FIG. 7 is a perspective view showing a further embodiment of packaging non-woven paper wipes contained on a roll;

FIG. 8 is a perspective view showing a liquid tight container for packaging the roll of FIG. 7, wherein the container includes a base and a removable lid;

FIG. 9 is a perspective view showing a further embodiment of the invention in the form of a dispensable cloth material pad;

FIG. 10 is a perspective view showing a stacked arrangement of the cloth material pads of FIG. 9 packaged within a liquid tight container including a base and removable lid;

FIG. 11 is a top perspective view illustrating an individual folded wipe formed of a non-woven paper material;

FIG. 12 is a top perspective view showing a liquid tight foil package for containing the individual wipe of FIG. 11;

FIG. 13 is a top perspective view showing the package of FIG. 12 partially torn to permit removal of the individual wipe contained therein; and

FIG. 14 is a perspective showing a plurality of foil packages of FIG. 12 packaged within a box including a hinged lid.

Like reference numerals refer to like parts throughout the several views of the drawings.

### DETAILED DESCRIPTION OF THE INVENTION

Non-woven materials pregnated with volatile aromatic compounds and antimicrobial compound of the present invention contain the essential components, as well as various optional components as indicated below.

### Aromatic Actives

The first essential component of the present invention is an aromatic active component. This aromatic active component comprises from about 0.1% to about 50%, preferably from about 1% to about 30% and most preferably from about 5% to about 15% of one or more volatile aromatic compounds selected from the group consisting of Menthol, Camphor, Eucalyptus oil, Cedarleaf Oil, Myristica Oil, Peppermint Oil, Lavender oil and mixtures thereof.

### Menthol

Menthol is a covalent organic compound made synthetically or obtained from peppermint or other mint oils. It is a waxy, crystalline substance, clear or white in color, which is solid at room temperature and melts slightly above. The main form of menthol occurring in nature is (−)-menthol, which is assigned the (1R,2S,5R) configuration. Menthol has local anesthetic and counterirritant qualities, and it is widely used to relieve minor throat irritation.

Most of menthol's uses are related to its stimulation of the skin's cold receptors. This property makes menthol produce a cooling effect when inhaled or applied to the skin. Similarly to the capsaicin chemical found in hot peppers, which stimulates heat receptors, menthol does not actually change the skin's temperature, but merely produces the sensation of temperature change.

Because of its cooling effect, menthol is used in products meant to relieve skin irritation, sore throat, or nasal congestion. It may be used to treat sunburn, fever, or muscle aches as well. In traditional Asian medicine, menthol may be prescribed for nausea, diarrhea, indigestion, headache, cold, or sore throat. When used as a supplement for health reasons, menthol is usually taken in the form of peppermint oil. Products that commonly contain menthol include toothpaste, cough drops, lip balm, mouthwash, gum, and cigarettes.

### Camphor

Camphor modern uses include as a plasticizer for cellulose nitrate, as a moth repellent, as an antimicrobial substance, in embalming, and in fireworks. Camphor crystals are also used to prevent damage to insect collections by other small insects. A form of anti-itch gel currently on the market uses camphor as its active ingredient. It is also used in medicine. Camphor is readily absorbed through the skin and produces a feeling of cooling similar to that of menthol and acts as slight local anesthetic and antimicrobial substance. Camphor is an active ingredient (along with menthol)

### Oil of Eucalyptus

Oil of eucalyptus is officially described as “a colorless or pale yellow liquid, having a characteristic, aromatic, somewhat camphoraceous odor, and a pungent, spicy, and cooling taste. It is soluble in 4 volumes of 70 percent. alcohol. Specific gravity: 0.905 to 0.925 at 25° C. (77° F.). Mix 2 mils of the Oil with 4 mils of glacial acetic acid and gradually add 3 mils of a saturated solution of sodium nitrite. When gently stirred, the mixture does not form crystals of phellandrene nitrite (other eucalyptus oils containing large amounts of phellandrene).

### Myristica Oil

Myristica used heavily in the perfumery and pharmaceutical industries. The oil is colourless or light yellow and smells and tastes of nutmeg. It contains numerous components of interest to the oleochemical industry, and is used as a natural food flavouring in baked goods, syrups, beverages, sweets etc. It replaces ground nutmeg as it leaves no particles in the food. The essential oil is also used in the cosmetic and pharmaceutical industries for instance in tooth paste and as major ingredient in some cough syrups. In traditional medicine nutmeg and nutmeg oil were used for illnesses related to the nervous and digestive systems. Myristicin and elemicin are believed to be the chemical constituents responsible for the subtle hallucinogenic properties of nutmeg oil. Other known chemical ingredients of the oil are α-pinene, sabinene, γ-terpinene and safrole.

### Peppermint Oil

Peppermint has a high menthol content, and is often used as a flavouring in tea, ice cream, confectionery, chewing gum, and toothpaste. The oil also contains menthone and menthyl esters. It is the oldest and most popular flavour of mint-flavoured confectionery. Peppermint can also be found in some shampoos and soaps, which give the hair a minty scent and produce a cooling sensation on the skin.

Peppermint, like many spices and herbs, is believed to have medicinal properties when consumed. It is said that it helps against upset stomachs, inhibits the growth of certain bacteria, and can help soothe and relax muscles when inhaled or applied to the skin. Other health benefits are attributed to the high manganese, vitamin C and vitamin A content; as well as trace amounts of various other nutrients such as fibre, iron, calcium, folate, potassium, tryptophan, magnesium, omega-3 fatty acids, riboflavin, and copper.

### Lavender Oil (Antiseptic and Anti-Inflammatory)

Lavender oil is an essential oil obtained by distillation from the flower spikes of certain species of lavender. Two forms are distinguished, Lavender Flower Oil, a colorless oil, insoluble in water, having a density of 0.885 (g/mL); and Lavender Spike Oil, a distillate from the herb Lavandula latifolia, having density 0.905. Lavender Flower Oil is a designation of the National Formulary and the British Pharmacopoeia. It is not a pure compound; it is a complex mixture of natural products. Lavender oil should never be taken internally.

An infusion of lavender is claimed to soothe and heal insect bites. Bunches of lavender are also said to ward off insects. If applied to the temples, lavender oil is said to soothe headaches. Lavender is frequently used as an aid to sleep and relaxation: Seeds and flowers of the plant are added to pillows, and an infusion of three flowerheads added to a cup of boiling water are recommended as a soothing and relaxing bedtime drink. Lavender oil (or extract of Lavender) is claimed to heal acne when used diluted 1:10 with water, rosewater, or witch hazel; it is also used in the treatment of skin burns and inflammatory conditions

### Pharmaceutical Actives

Pharmaceutical actives useful in the present invention include any chemical material or compound suitable for topical administration; however, such drugs should be included so as not to interfere with the stability of the composition. These actives are present at a level from about 0.05% to about 20%. Such substances include, but are not limited to antibiotics, wound healing agents, vitamins, antiviral agents, analgesics, anti-inflammatory agents, antipuritics, antipyretics, anesthetic agents, antifungals, antimicrobials and mixtures thereof.

A safe and effective amount of an anti-inflammatory agent may be added to the compositions of the present invention, preferably from about 0.05% to about 10%, more preferably from about 0.5% to about 5%, of the composition. The exact amount of anti-inflammatory agent to be used in the compositions will depend on the particular anti-inflammatory agent utilized since such agents vary widely in potency.

### Antiviral Drugs

Antiviral drugs are a class of medication used specifically for treating viral infections. Like antibiotics, specific antivirals are used for specific viruses. Antiviral drugs are one class of antimicrobials, a larger group which also includes antibiotic, antifungal and antiparasitic drugs. They are relatively harmless to the host, and therefore can be used to treat infections. They should be distinguished from viricides, which actively deactivate virus particles outside the body.

Most of the antivirals now available are designed to help deal with HIV; herpes viruses, best known for causing cold sores and genital herpes, but actually causing a wide range of diseases; the hepatitis B and C viruses, which can cause liver cancer; and influenza A and B viruses. Researchers are now working to extend the range of antivirals to other families of pathogens.

The emergence of antivirals is the product of a greatly expanded knowledge of the genetic and molecular function of organisms, allowing biomedical researchers to understand the structure and function of viruses, major advances in the techniques for finding new drugs, and the intense pressure placed on the medical profession to deal with the human immunodeficiency virus (HIV), the cause of the deadly acquired immunodeficiency syndrome (AIDS) pandemic.

Almost all anti-microbials, including anti-virals, are subject to drug resistance as the pathogens evolve to survive exposure to the treatment. As of 2007, only smallpox has been successfully eradicated, and Poliomyelitis eradication is still underway. Both of these efforts are using vaccines.

### Antimicrobial Agent

### Benzethonium Chloride

Benzethonium chloride is a synthetic quaternary ammonium, surfactant, antiseptic, and anti-infective compound used as a topical antimicrobial agent in cosmetics and personal care products like anti-itch ointments and antibacterial moist towelettes and wipes. Benzothonium chloride is also used is the food industry as a disinfectant and preservative.

### Phenoxyethanol

Phenoxyethanol is an organic chemical compound, a glycol ether often used in dermatological products such as skin creams. It is a colorless oily liquid. It is a bactericide (usually used in conjunction with quaternary ammonium compounds), often used in place of sodium azide in biological buffers as 2-phenoxyethanol is less toxic and non-reactive with copper and lead. It is also used as a fixative for perfumes, an insect repellent, a topical antiseptic, a solvent for cellulose acetate, some dyes, inks, and resins, in preservatives, pharmaceuticals, and in organic synthesis. It is moderately soluble in water.

### Methylparaben (Mold-Inhibitor)

Methylparaben, also methyl paraben, one of the parabens, has formula CH₃(C₆H₄(OH)COO). It is the methyl ester of p-hydroxybenzoic acid. It is a mold-inhibitor and a popular preservative for food and cosmetics.

### Propylparaben (Preservative)

Propylparaben, the propyl ester of p-hydroxybenzoic acid, occurs as a natural substance found in many plants and some insects, although it is manufactured synthetically for use in cosmetics, pharmaceuticals and foods. It is a preservative typically found in many water-based cosmetics, such as creams and lotions and some bath products.

### 2-BROMO-2-NITROPROPANE-1,3-DIOL

2-bromo-2-nitropropane-1,3-diol is antimicrobial chemical compound call Bronopol, Bronopol was invented by The Boots Company PLC, Nottingham, England in the early 1960s and first applications were as a preservative for pharmaceuticals. Bronopol's low mammalian toxicity (at in-use levels) and exceptional activity against bacteria (especially the troublesome Gram-negative species) ensured that it became popular as a preservative in many consumer products such as shampoos and cosmetics.

Bronopol was subsequently taken up as an effective antimicrobial in many industrial environments such as paper mills, oil exploration and production facilities, as well as cooling water disinfection plants.

Non-steroidal anti-inflammatory drugs, usually abbreviated to NSAIDs, are drugs with analgesic, antipyretic and anti-inflammatory effects—they reduce pain, fever and inflammation. The term “non-steroidal” is used to distinguish these drugs from steroids, which (among a broad range of other effects) have a similar eicosanoid-depressing, anti-inflammatory action. As analgesics, NSAIDs are unusual in that they are non-narcotic. NSAIDs are sometimes also referred to as non-steroidal anti-inflammatory agents/analgesics (NSAIAs). The most prominent members of this group of drugs are aspirin and ibuprofen. Paracetamol (acetaminophen) has negligible anti-inflammatory activity, and is strictly speaking not an NSAID.

Steroidal anti-inflammatory agents, including but not limited to, corticosteroids such as beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednyl idene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, hydrocortisone, hydroxyltriamcinolone, alpha-methyl dexamethasone, dexamethasone-phosphate, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use in the present invention is Beclometasone dipropionate

### Beclometasone Dipropionate

Beclometasone dipropionate (INN modified) or beclomethasone dipropionate (USAN, former BAN), also referred to as beclometasone (INN), is a potent Class A glucocorticoid steroid drug. In the form of an inhaler (e.g. Becotide), a wide number of brands of which are available, it is used for the prophylaxis of asthma. As a nasal spray (e.g. Beconase, Vancenase), it is used for the treatment of rhinitis (e.g. hayfever) and sinusitis. In some instances it is used by oral pathologists in the treatment of unusually severe canker sores.

### Non-Steroidal Anti-Inflammatory Agents (NSAIDS)

Specific non-steroidal anti-inflammatory agents useful in solution composition of the present invention include, but are not limited to: the oxicams, such as piroxicam, isoxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and the pyrazoles, such as phenybutazone, oxyphenbutazone, feprazone, azapropazone, and trimethazone

Solution Mixtures of these non-steroidal anti-inflammatory agents may also be employed, as well as the pharmaceutically-acceptable salts and esters of these agents. For example, etofenamate, a flufenamic acid derivative, is particularly useful for topical application. Of the nonsteroidal anti-inflammatory agents, ibuprofen, naproxen, flufenamic acid, mefenamlc acid, meclofenamic acid, piroxicam and felbinac are preferred; ibuprofen, and naproxen are most preferred.

### Naproxen (Non-Steroidal Anti-Inflammatory)

Naproxen (INN) (IPA: is a non-steroidal anti-inflammatory drug (NSAID) commonly used for the reduction of moderate to severe pain, fever, inflammation and stiffness caused by conditions such as osteoarthritis, rheumatoid arthritis, psoriatic arthritis, gout, ankylosing spondylitis, injury (like fractures), menstrual cramps, tendinitis, bursitis, and the treatment of primary dysmenorrhea. Naproxen and naproxen sodium are marketed under various trade names including: Aleve, Anaprox, Naprogesic, Naprosyn, Naprelan, Synflex.

### Anesthetic or Antipruritic

Useful anesthetic or antipruritic drugs are selected from the group consisting of lidocaine, lidocaine hydrochloride, bupivacaine hydrochloride, chlorprocaine hydrochloride, dibucaine hydrochloride, etidocaine hydrochloride, mepivacaine hydrochloride, Myristica Oil, tetracaine, tetracaine hydrochloride, dyclonine hydrochloride and hexylcaine hydrochloride, benzocaine, Oil of eucalyptus, benzyl alcohol, butamben picrate, camphor, camphorated metacresol, dibucaine, dibucaine hydrochloride, dimethisoquin hydrochloride, diphenhydramine hydrochloride, juniper tar, menthol, Peppermint Oil, phenol, phenolate sodium, pramoxine hydrochloride, resorcinol and mixtures thereof. Of the anesthetic or antipruritic drugs Menthol, Camphor, Eucalyptus oil, Cedarleaf Oil, Myristica Oil, Peppermint Oil, are most preferred.

### Aromatics

Various other non-active aromatic components (e.g., esters and aldehydes) may also be used. These aromatics include, for example, benzaldehyde (cherry, almond); citral (lemon, lime); neral; decanal (orange, lemon); aldehyde C-8, aldehyde C-9 and aldehyde C-12 (citrus fruits); tolyl aldehyde (cherry, almond); 2,6-dimethyl-octanal (green fruit); and 2-dodecenal (citrus, mandarin). Mixtures of these aromatics can also be used.

### Other Optional Components

A variety of additional ingredients may be added to the emulsion compositions of the present invention. These additional ingredients include various substantivity of the formulation, preservatives for maintaining the antimicrobial integrity of the compositions, antioxidants, and agents suitable for aesthetic purposes such as fragrances, Thymol, Disodium EDTA (stabilizer) and Nutmeg (Essential oils)

### Essential Oils

The essential oil is obtained by the steam distillation of ground nutmeg and is used heavily in the perfumery and pharmaceutical industries. The oil is colourless or light yellow and smells and tastes of nutmeg. It contains numerous components of interest to the oleochemical industry, and is used as a natural food flavouring in baked goods, syrups, beverages, sweets etc. It replaces ground nutmeg as it leaves no particles in the food. The essential oil is also used in the cosmetic and pharmaceutical industries for instance in tooth paste and as major ingredient in some cough syrups. In traditional medicine nutmeg and nutmeg oil were used for illnesses related to the nervous and digestive systems. Myristicin and elemicin are believed to be the chemical constituents responsible for the subtle hallucinogenic properties of nutmeg oil.

### Thymol (Stabilising Agent)

Thymol is a monoterpene phenol derivative of cymene, C₁₀H₁₃OH, isomeric with carvacrol, found in oil of thyme, and extracted as a white crystalline substance of a pleasant aromatic odor and strong antiseptic properties. It is also called “hydroxy cymene”. It is used as a preservative in halothane, an anaesthetic.

The pH of the compositions is preferably from about 5 to about 9, more preferably from about 6.5 to about 8.

The amount of active components and frequency of treatment will vary widely depending upon the individual.

Preferably, the wipes of this invention, which are held to the mouth and nose while gently sniffing through nostrils to help smooth breathing and comfort, can also be used to wipe the nose. The pregnanted wipe or pad composition is sniffed as needed to treat cough, cold, cold-like and/or flu symptoms. The amount of actives and frequency use can vary widely, depending upon personal needs or individual, but it is suggested as an example that application ranges from about one to four treatments per hour or as needed.

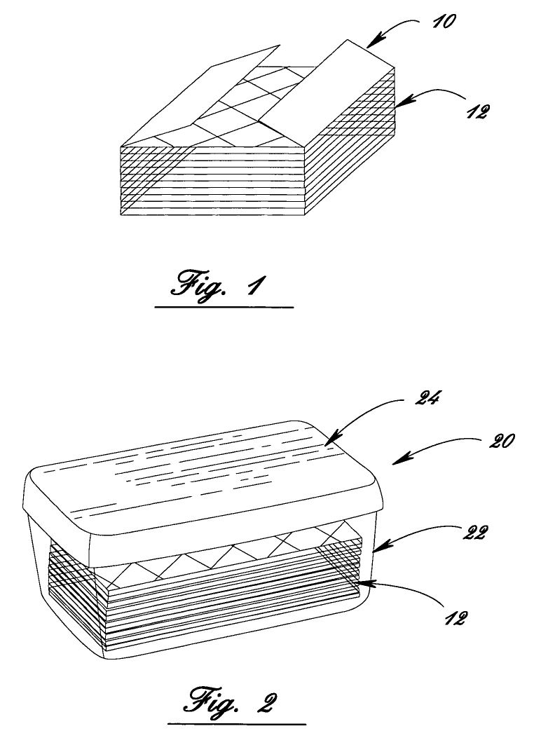

Referring to the drawings, several embodiments of the sheet substrate and packaging methods are shown. FIG. 1 shows a first preferred embodiment of the sheet substrate in the form of a wipe manufactured from a non-woven paper material, and is generally indicated as 10. The individual sheets of wipes 10 are folded and arranged in a stacked array 12 for packaging in accordance with several packaging methods, as shown in FIGS. 2-6.

FIG. 2 illustrates a first example of container for packaging the stack array 12 of wipes 10 that have been impregnated with one or more of the compositions disclosed herein, including one or more of the topical aromatic releasing compositions. Specifically, the container, generally indicated as 20, includes a base tub 22 portion and a removable lid 24. The removable lid 24 attaches to an open top of the tub 22 to provide a sealed, water tight environment within the container in order to preserve the compositions impregnated within the wipes 10. Both the tub 22 and the lid 24 are formed of a suitable plastic and/or rubber and plastic composite material.

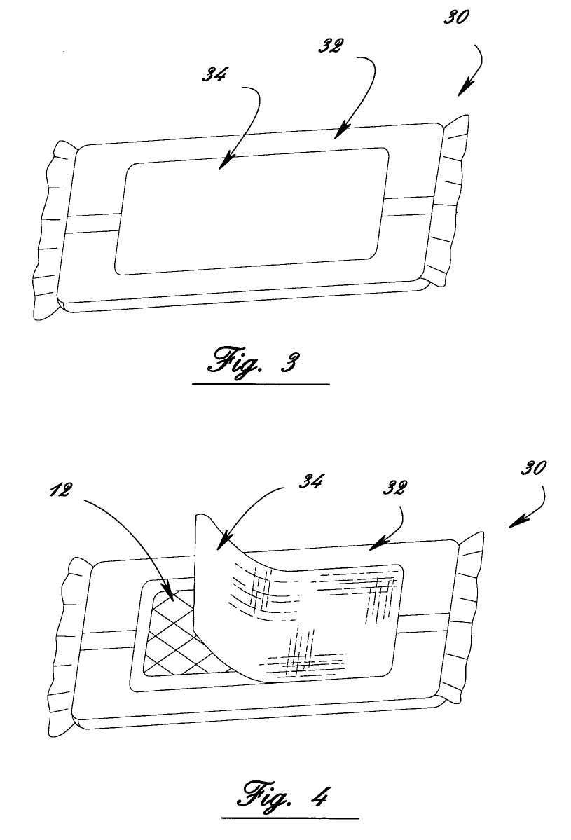

FIGS. 3 and 4 illustrate a further embodiment of the packaging method of the invention directed to a travel pack 30 defined by a liquid impervious flexible envelope 32 including a peel-away resealable cover 34 for opening the package in order to remove the individual wipes 10 from the stacked array 12 contained therein. The peel-away resealable cover 34 is movable to cover the opening of the envelope after removal of one or more wipes, and reseals the interior of the package to preserve the impregnated compositions.

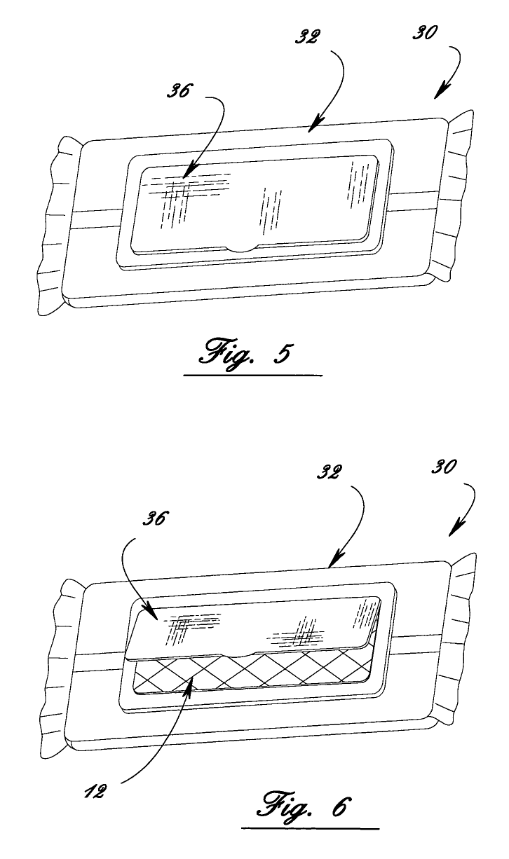

FIGS. 5 and 6 illustrate a further embodiment of the travel pack 30, wherein the flexible envelope 32 includes a rigid cover 36 that is hinged to the envelope 32 and operable between a closed, sealed position and an open position to permit individual removal of one or more of the wipes 10 from the stacked array 12 contained within the envelope package 32.

FIGS. 7 and 8 illustrate a further embodiment of sheet substrate and packaging method of the present invention, wherein the sheet substrate is provided in the form of a non-woven paper product 40 wound on a roll 42, similar to toilet tissue. The roll 42 is packaged within a container 50, as seen in FIG. 8. The container 50 includes a base portion 52 within an interior sized and configured for receipt of the roll 42 therein. A removable lid 54 attaches to the base 52 to provide a water tight seal in order to preserve the compositions impregnated in the sheet substrate 40.

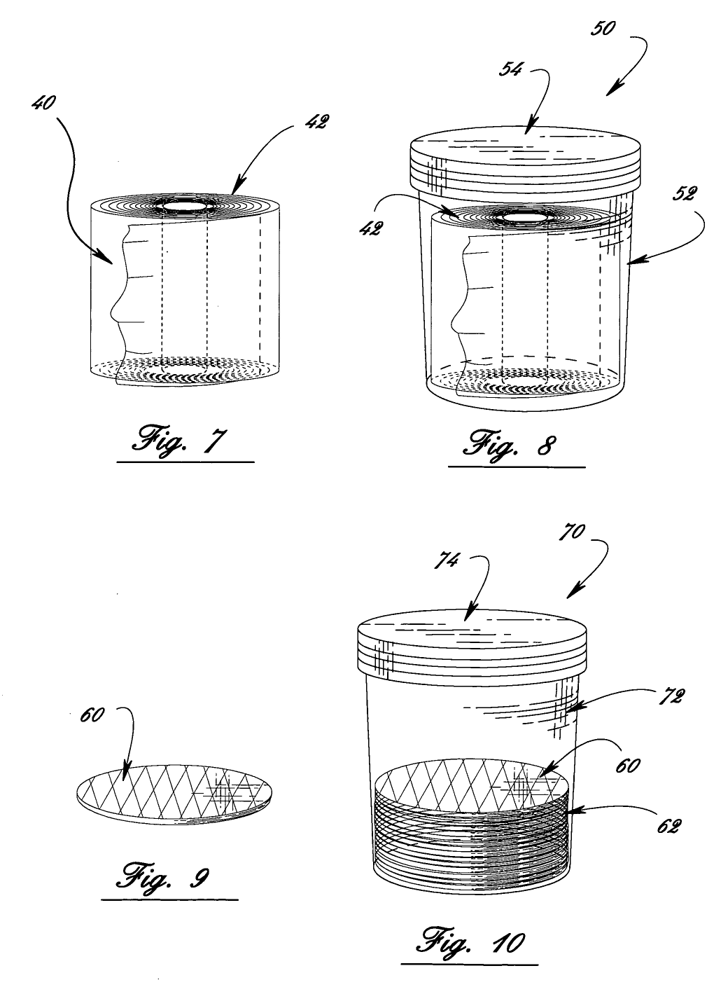

FIGS. 9 and 10 illustrate yet a further embodiment of the sheet substrate and packaging method of the present invention, wherein the sheet substrate is provided in the form of a dispensable cloth pad 60. A plurality of pads 60 are packaged in a stacked array 62 and packaged within a cylindrical container 70, including a base 72 and a removable lid 74. The lid 74 attaches to the base 72 to provide a water tight seal in order to preserve the compositions impregnated within the pads 60.

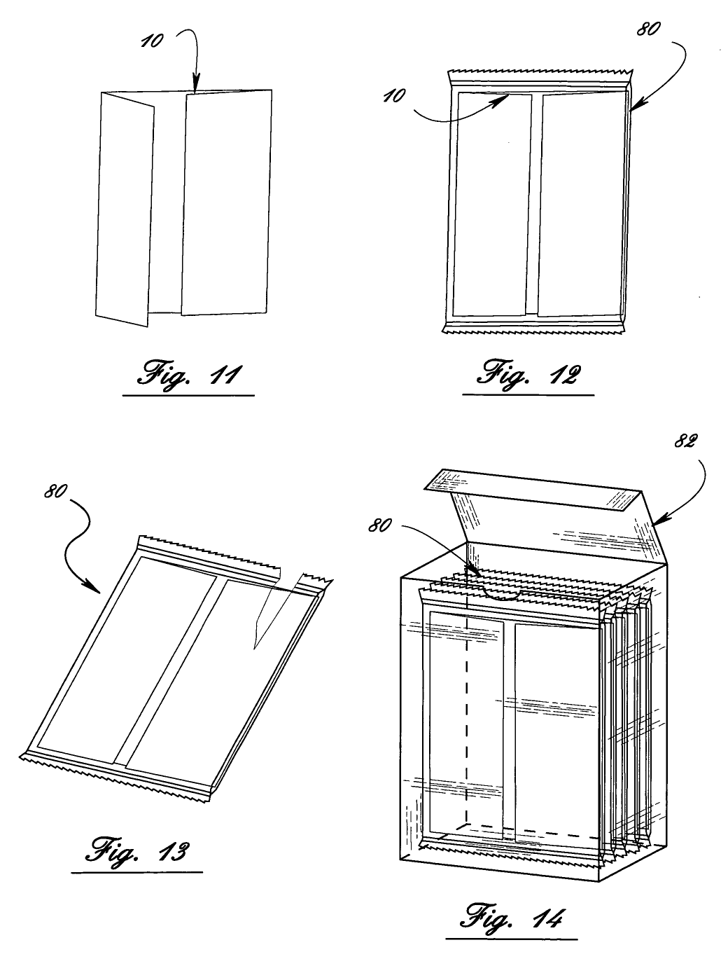

FIGS. 11 through 14 illustrate a further embodiment of the sheet substrate and packaging method of the present invention directed to a single sheet 10 of a non-woven paper material that is impregnated with one or more compositions disclosed herein. As seen in FIG. 12, the single sheet substrate 10 is packaged within a liquid impervious flexible material package, such as a foil package 80 that is air tight and water tight in order to preserve the liquid compositions impregnated in the sheet substrate 10. To open the package 80, an end is torn, as shown in FIG. 13, thereby enabling removal of the sheet substrate 10 for a one time use. Preferably, the sheet substrate is in the form of a hand wipe or tissue. As seen in FIG. 14, a plurality of the packages 80 containing the individual wipes 10 are packaged in a box 82 having a top flap lid that opens to facilitate individual removal of the packages 80.

The resultant formulation is suitable for a wide variety of therapeutic agents such as anti-inflammatory agents, aromatics, antifungal agents, antibiotics, anesthetics, etc., which are administered for their known indications. Advantageously, the aromatic wipes is stable, does not leave any greasy residue on skin or face after use, and appears to enjoy user preference when compared to other conventional aromatic products. The following example will serve to further typify the nature of the invention but should not be construed as a limitation on the scope there which is defined solely by the appended claims.

### EXAMPLE 1

### Aromatics Spiking Solution

The following ingredients are thoroughly blended:

The following examples further describe and demonstrate embodiments within the scope of the present invention. The examples are given solely for the purpose of illustration and are not to be construed as limitations of the present invention, as many variations thereof are possible without departing from the spirit and scope of the invention.

Ingredients are identified by chemical or CTFA name.

| Ingredients | W/W 100% |
|---|---|
| Liquid paraffin | 25.00 |
| Menthol | 2.81 |
| Camphor | 5.23 |
| Eucalyptus Oil | 1.34 |
| Cedarleaf Oil | 0.44 |
| Myristica Oil | 0.69 |
| Peppermint Oil | 1.50 |
| Lavender Oil | 2.10 |
| Methyl Salicylate | 0.20 |
| Naproxen | 0.10 |
| Nutmeg Oil | 1.00 |
| Thymol | 0.09 |
| Beclometasone dipropionate | 0.02 |
| Benzethonium chloride | 0.05 |
| q.s. Liquid paraffin | 59.43 |

In a suitable size container, (preferable container and mixer are glass bottle, Teflon container, 316 stainless steel container, mix with Teflon Magnet or 316 stainless steel mixer for example, a Lightnin' mixer and or a magnetic mixer) large enough to produce the total Lot batch add Liquid paraffin while mixing add menthol, camphor, eucalyptus oil, cedarleaf oil, myristica oil, Peppermint Oil, Lavender Oil, Methyl Salicylate, Naproxen, Nutmeg Oil, Thymol, Beclometasone dipropionate, Benzethonium chloride and q.s. with Liquid paraffin mix slowly but gentle heat to about 75° C., mix for 120 minutes

### EXAMPLE 2

### Aromatics Solution

Use of approximately Five to Ten grams of the composition from Aromatics Spiking Solution in example 1 is useful for graduating strength or potency of the topical application for relief from cough, cold, cold-like and/or flu symptoms.

The following ingredients are thoroughly blended:

Ingredients are identified by chemical or CTFA name.

| Ingredients | W/W 100% |
|---|---|
| Deionized Water | 25.00 |
| Cetearyl Isononanoate | 0.03 |
| Ceteareth-20 | 0.01 |
| Cetyl Alcohol | 2.25 |
| Glyceryl Stearate | 1.00 |
| Glycerin | 10.00 |
| Cetyl Palmitate | 0.03 |
| Ceteareth-12, | 0.01 |
| Lauryl Glucose | 0.20 |
| Lauryl Glucoside | 0.05 |
| Aromatics Spiking Solution | 10.20 |
| Isopropyl Alcohol | 2.50 |
| Phenoxyethanol | 0.30 |
| Methylparaben | 0.25 |
| Propylparaben | 0.10 |
| 2-bromo-2-nitropropane-1,3-diol | 0.20 |
| Sodium Citrate | q.s. |
| Citric Acid | q.s. |
| Deionized Water | 32.50 |

In a suitable size container, (preferable container and mixer are glass bottle, Teflon container, 316 stainless steel container, mix with Teflon Magnet or 316 stainless steel mixer for example, a Lightnin' mixer and or a magnetic mixer) large enough to produce the total Lot batch add Deionized Water while mixing add Cetearyl Isononanoate, Ceteareth-20, Cetyl Alcohol, Glyceryl Stearate, Glycerin, Cetyl Palmitate, Ceteareth-12, Lauryl Glucose, Lauryl Glucoside, Aromatics Spiking Solution, while mixing add Isopropyl Alcohol, Phenoxyethanol, Methylparaben, Propylparaben, 2-bromo-2-nitropropane-1,3-diol While mixing, add Fragrance while mixing, Measure the pH.

pH: specification is between 6.05±0.5

The pH scale, Measures how acid or alkaline is in the Solution. Adjust the pH as needed Adjusting the pH:

If the pH is below 6.05 Adjust the pH by using 10% Sodium Citrate Solution

If the pH is above 6.5

Adjust the pH by using 10% Citric Acid Solution.

Mix for 60 minutes. While mixing q.s. with Deionized Water at room temperature mix for 30 minutes, filter through 0.5 micron filter.

Disperse uniformly the aromatic solution into Wipe paper materials (Non-woven) and or a dispensable cloth materials (gauze or a thin fabric of silk linen, or cotton materials), Paper Pad, Cloth or Gauze Pad pack in plastic film, pack in a Flat or round Can or glass Bottle, in a plastic container or comparable paper product.

### EXAMPLE 3

### Non-Alcohol Aromatics Solution

Use of approximately Five grams of the composition from Aromatics Spiking Solution in example 1 is useful for graduating strength or potency of a non-Alcohol topical application for relief from cough, cold, cold-like and/or flu symptoms.

The following ingredients are thoroughly blended:

Ingredients are identified by chemical or CTFA name.

| Ingredients | W/W 100% |
|---|---|
| Deionized Water | 25.00 |
| Vegetable Oil | 0.20 |
| Dicaprylyl Carbonate | 0.10 |
| Glyceryl Oleate | 0.15 |
| Glycerin | 0.10 |
| Lauryl Glucoside | 0.05 |
| Polyglyceryl-2 Dipolyhydroxystearate | 0.03 |
| Aromatics Spiking Solution | 5.81 |
| Phenoxyethanol | 0.30 |
| Methylparaben | 0.25 |
| Propylparaben | 0.10 |
| 2-bromo-2-nitropropane-1,3-diol | 0.20 |
| Fragrance | q.s. |
| Sodium Citrate | q.s. |
| Citric Acid | q.s. |
| Deionized Water | 32.50 |

In a suitable size container, (preferable container and mixer are glass bottle, Teflon container, 316 stainless steel container, mix with Teflon Magnet or 316 stainless steel mixer for example, a Lightnin' mixer and or a magnetic mixer) large enough to produce the total Lot batch add Deionized Water while mixing add Vegetable Oil, Dicaprylyl Carbonate, Glyceryl Oleate, Glycerin, Lauryl Glucoside, Polyglyceryl-2 Dipolyhydroxystearate, Sodium Citrate, Citric Acid, Aromatics Spiking Solution, while mixing add Phenoxyethanol, Methylparaben, Propylparaben, 2-bromo-2-nitropropane-1,3-diol While mixing, add Fragrance while mixing, Measure the pH. pH: specification is between 6.05±0.5

The pH scale, Measures how acid or alkaline is in the Solution. Adjust the pH as needed Adjusting the pH:

If the pH is below 6.05 Adjust the pH by using 10% Sodium Citrate Solution

If the pH is above 6.5

Adjust the pH by using 10% Citric Acid Solution.

Mix for 60 minutes. While mixing q.s. with Deionized Water at room temperature mix for 30 minutes, filter through 0.5 micron filter.

Disperse uniformly the aromatic solution into Wipe paper materials (Non-woven) and or a dispensable cloth materials (gauze or a thin fabric of silk linen, or cotton materials), Paper Pad, Cloth or Gauze Pad pack in plastic film, pack in a Flat or round Can or glass Bottle, in a plastic container or comparable paper product.

What is claimed is:

1. An article of manufacture for providing relief from cold, allergies, sinus and symptoms associated with respiratory disorders, said article comprising:
a sheet substrate impregnated with an aromatic releasing composition containing one or more volatile aromatic compounds selected from the group consisting of: Menthol; Camphor; Eucalyptus oil; Cedarleaf Oil; Myristica Oil; Peppermint Oil; Lavender oil; and mixtures thereof, wherein said composition is in the form of an oil-in-water emulsion.

2. The article of claim 1 wherein said sheet substrate comprises a non-woven paper wipe.

3. The article of claim 2 wherein said sheet substrate is impregnated with an antimicrobial composition selected from the group consisting of: between 0.01% and 30% of pseudomonas aeruginosa; Escherichia coli; proteus vulgaris; salmonella entiritidis; staphylococcus aureus; enterococcus faecium; bacillus cereus; β-lactam drugs; quinolone drugs; ciprofloxacin; norfloxacin; tetracycline; erythromycin; amikacin; triclosan; doxycycline; capreomycin; chlorhexidine; chlortetracycline; oxytetracycline; clindamycin; ethambutol; metronidazole; pentamidine; gentamicin; kanamycin; lineomycin; methacycline; methenamine; minocycline; neomycin; netilmicin; paromomycin; streptomycin; tobramycin; miconazole; Phenoxyethanol; Methylparaben; Propylparaben; 2-bromo-2-nitropropane-1,3-diol; amanfadine; pharmaceutically-acceptable salts and mixtures thereof.

4. The article of claim 2 wherein said sheet substrate is impregnated with an anesthetic and antipruritic composition selected from the group consisting of: lidocaine; lidocaine hydrochloride; bupivacaine hydrochloride; chlorprocaine hydrochloride; dibucaine hydrochloride; etidocaine hydrochloride; mepivacaine hydrochloride; tetracaine; tetracaine hydrochloride; dyclonine hydrochloride and hexylcaine hydrochloride; benzocaine; benzyl alcohol; butamben picrate; camphor; camphorated metacresol; dibucaine; dibucaine hydrochloride; dimethisoquin hydrochloride; diphenhydramine hydrochloride; juniper tar; menthol; phenol; phenolate sodium; pramoxine hydrochloride; resorcinol and mixtures thereof.

5. The article of claim 2 wherein said sheet substrate is impregnated with a non-steroidal anti-inflammatory composition selected from the group consisting of: aspirin; acetaminophen; ibuprofen; naproxen; benoxaprofen; flurbiprofen; fenoprofen; fenbufen; ketoprofen; indoprofen; pirprofen; carprofen; oxaprozin; pranoprofen; miroprofen; tioxaprofen; suprofen; alminoprofen; tiaprofenic acid; fluprofen; and bucloxic acid and mixtures thereof.

6. The article of claim 2 wherein said sheet substrate is impregnated with an emollient selected from the group consisting of: Lauryl Glucoside; Cetearyl Isononanoate; ceteareth-20; cetearyl alcohol; glyceryl stearate; glycerin; cetyl palmitate; ceteareth-12; lauryl glucose carboxylate; deionized water; and mixtures thereof.

7. The article of claim 2 wherein said sheet substrate is impregnated with vitamins selected from the group consisting of: vitamin A and derivatives thereof; ascorbic acid; vitamin B; biotin; panthothenic acid; vitamin D; vitamin E; and mixtures thereof.

8. The article of claim 2 wherein said sheet substrate is further impregnated with an antimicrobial microorganism mold selected from the group consisting of: aspergillus niger; pencillium purpurogenum; and mixtures thereof.

9. A method for treatment of cough, cold, cold-like and/or flu symptoms comprising administering the aromatic releasing composition of claim 1 by applying the sheet substrate to the nose and face for inhaled delivery of the aromatic releasing composition.

10. A method for discouraging infection with virus comprising administering the antimicrobial composition of claim 3 by applying the sheet substrate to the nose and face for inhaled delivery of the antimicrobial composition.

11. A method for treatment of cough, cold, cold-like and/or flu symptoms comprising administering the non-steroidal anti-inflammatory composition of claim 5 by applying the sheet substrate to the nose and face for inhaled delivery of the non-steroidal anti-inflammatory composition.

12. The article of claim 1 wherein said sheet substrate comprises a dispensable cloth material.

13. The article of claim 12 wherein said sheet substrate is impregnated with an antimicrobial composition selected from the group consisting of: between 0.01% and 30% of pseudomonas aeruginosa; Escherichia coli; proteus vulgaris; salmonella entiritidis; staphylococcus aureus; enterococcus faecium; bacillus cereus; β-lactam drugs; quinolone drugs; ciprofloxacin; norfloxacin; tetracycline; erythromycin; amikacin; triclosan; doxycycline; capreomycin; chlorhexidine; chlortetracycline; oxytetracycline; clindamycin; ethambutol; metronidazole; pentamidine; gentamicin; kanamycin; lineomycin; methacycline; methenamine; minocycline; neomycin; netilmicin; paromomycin; streptomycin; tobramycin; miconazole; Phenoxyethanol; Methylparaben; Propylparaben; 2-bromo-2-nitropropane-1,3-diol; amanfadine; pharmaceutically-acceptable salts and mixtures thereof.

14. The article of claim 12 wherein said sheet substrate is impregnated with an anesthetic and antipruritic composition selected from the group consisting of: lidocaine; lidocaine hydrochloride; bupivacaine hydrochloride; chlorprocaine hydrochloride; dibucaine hydrochloride; etidocaine hydrochloride; mepivacaine hydrochloride; tetracaine; tetracaine hydrochloride; dyclonine hydrochloride and hexylcaine hydrochloride; benzocaine; benzyl alcohol; butamben picrate; camphor; camphorated metacresol; dibucaine; dibucaine hydrochloride; dimethisoquin hydrochloride; diphenhydramine hydrochloride; juniper tar; menthol; phenol; phenolate sodium; pramoxine hydrochloride; resorcinol and mixtures thereof.

15. The article of claim 12 wherein said sheet substrate is impregnated with a non-steroidal anti-inflammatory composition selected from the group consisting of: aspirin; acetaminophen; ibuprofen; naproxen; benoxaprofen; flurbiprofen; fenoprofen; fenbufen; ketoprofen; indoprofen; pirprofen; carprofen; oxaprozin; pranoprofen; miroprofen; tioxaprofen; suprofen; alminoprofen; tiaprofenic acid; fluprofen; and bucloxic acid and mixtures thereof.

16. The article of claim 12 wherein said sheet substrate is impregnated with an emollient selected from the group consisting of: Lauryl Glucoside; Cetearyl Isononanoate; ceteareth-20; cetearyl alcohol; glyceryl stearate; glycerin; cetyl palmitate; ceteareth-12; lauryl glucose carboxylate; deionized water; and mixtures thereof.

17. The article of claim 12 wherein said sheet substrate is impregnated with vitamins selected from the group consisting of: vitamin A and derivatives thereof; ascorbic acid; vitamin B; biotin; panthothenic acid; vitamin D; vitamin E; and mixtures thereof.

18. The article of claim 12 wherein said sheet substrate is further impregnated with an antimicrobial microorganism mold selected from the group consisting of: aspergillus niger; pencillium purpurogenum; and mixtures thereof.
